(19) United States
(12) Patent Application Publication
(10) Pub. No.: US 2007/0119712 A1
(43) Pub. Date: May 31, 2007

(54) Electrophoretic separation of amphoteric molecules

(75) Inventor: Detlev Hadbawnik, Waldbronn (DE)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(73) Assignee: Agilent Technologies, Inc., Palo Alto, CA (US)

(21) Appl. No.: 10/556,196

(22) PCT Filed: Sep. 24, 2003
(86) PCT No.: PCT/EP03/50651

§ 371 Date: Feb. 5, 2007

(51) Int. Cl. C07K 1/28; B01D 57/02; G01N 27/00

(52) U.S. Cl. 204/459; 204/548; 204/610; 204/644

(57) ABSTRACT

Disclosed is a separation of amphoteric molecules according to their isoelectric points, wherein a first separation according to the isoelectric points of the molecules in one ore more first strip-like separation media located on a first carrier is carried out. First separation media have a first pl range.

### 1. FIELD OF THE INVENTION

The present invention relates to electrophoretic separation of amphoteric molecules according to their isoelectric points.

### 2. DISCUSSION OF THE BACKGROUND ART

Proteomics studies, which are used to analyze a plurality of proteins present in a cell, require fast and high resolution separation techniques in order to separate and analyze single protein species in a relatively short period of time. A preliminary sequencing of the human genome sequence e.g. revealed that roughly 30 000 to 70 000 open reading frames (ORF) are present in the human genome. Between 100 000 and two million proteins are believed to be expressed in human cells, suggesting that a high rate of messenger RNA splicing and post-translational modifications (PTMs) might be responsible for the plethora of proteins. Post-translational modifications, which can comprise the modification of amino-acids for example proline to hydroxyproline or the linking of carbohydrates to amino-acid side chains, are hard to resolve and analyze.

A state of the art separation technique is the two-dimensional gel electrophoresis. This separation technique involves the separation by isoelectric point in a first dimension by conducting an isoelectric focusing and a separation by size in the second dimension by performing a sodium dodecyl sulphate polyacrylamide gel electrophoresis (SDS-PAGE). This separation technique is able to resolve up to 10 000 components for every gel. One major disadvantage of the two-dimensional gel electrophoresis (2D SDS-PAGE) is its labor-intensive and therefore time-consuming handling.

This especially involves the handling of the first-dimension isoelectric focusing gel and the transfer of the pl gradient to the SDS-PAGE gel and subsequent staining and destaining steps. Another disadvantage of the 2D SDS-PAGE is the gel's modest sample capacity preventing the loading of sufficient protein amounts to be able to visualize low abundance proteins or PTMs. Therefore low abundance proteins or PTMs, which are often regulatory proteins, involved in certain diseases, are frequently masked by high abundance proteins in 2D SDS-PAGE.

### SUMMARY OF THE INVENTION

It is an object of the present invention to provide an improved separation technique. This is solved by the independent claims. Favorable embodiments are subjects of further claims. Thus, preferred embodiments provide a fast separation technique, which can be used for example in proteomic studies, and can also enable to visualize low abundance proteins.

In one embodiment, a method for electrophoretic separation of amphoteric molecules according to their isoelectric points comprises:
A) Carrying out a first separation according to the isoelectric points of the amphoteric molecules in one ore more first strip-like separation media located on a first carrier, wherein the first separation media have a respective first pl range.

The method provides a fast and easy to handle procedure for separation of a large amount of amphoteric molecules, for example polypeptides or oligopeptides, because many separation media, which are located on the same carrier can easily and quickly be processed at the same time in a single step. This allows a relatively short separation time of less than 24 hours.

In another variant of the method, the amphoteric molecules separated in A) are isolated from the first strip-like separation media in a D1).

A two-step separation procedure, which apart from A) additionally comprises the following:
B) Transferring the amphoteric molecules separated in A) to a plurality of second strip-like separation media located on a second carrier, wherein the second separation media have a respective second pl range different from the first pl range,
C) carrying out a second separation of the molecules in the plurality of second separation media according to isoelectric points,

D2) isolating the molecules from the second strip-like separation media.

Due to the plurality of the first and second separation media on first and second carriers, this variant of the method provides the possibility to load high amounts of molecules onto the separation media, therefore enabling the separation and subsequent detection of low-abundance proteins or PTMs of high interest. Due to the two different isoelectric separations in A) and C), this method also provides a good high resolution separation of the molecules according to their charges (isoelectric points). The simultaneous processing of a large number of samples furthermore allows a high throughput separation and analysis of the entire complement of proteins expressed in a cell or tissue.

In a preferred embodiment, first separation media each having the same pl range are used and second separation media each having different starting and end points for their pl ranges are used. Due to the fact that the first separation media all have the same pl range and are processed in a single step, it is possible to load aliquots of the same sample on each of the first separation media advantageously in an automated method. The second separation media can then provide a further second separation of the molecules already pre-separated in A).

In another preferred embodiment, the first and second separation media are selected in such a way that the respective pl range of the first separation media is larger than the respective pl range of the second separation media. For example the first separation media can have a maximum pl range from approximately 1 to 12, whereas the second separation media can have a maximum pl range of 1 or even smaller than 1.

This variant provides a two-step isoelectric focusing procedure with a first so-called wide range separation for example over the full pl range of a complement of proteins expressed in a cell or a tissue and a second so-called narrow range separation. During the narrow range separation molecules, which were already pre-separated in the first separation A) and roughly have the same isoelectric point range (for example isoelectric point range between 3 and 4) are subjected to a narrow range separation in C), where the proteins are separated according to their isoelectric points with a higher resolution. For example, it is possible to isolate the molecules roughly having the same pl range, for example between 3 and 4 from the first separation media with pl ranges from 3 to 10 and subject these molecules to a second narrow range separation on a second separation medium with a pl range roughly between 3 and 4, therefore allowing a higher resolution of separation in the second separation C). This two-step isoelectric focusing (IEF) procedure with the first wide range separation and the second narrow range separation furthermore allows the separation and subsequent identification of proteins with post-translational modifications, which differ in their pl by about 0.1 to 0.05 pl units. Therefore, this allows the identification of even very small functional differences between otherwise similar proteins.

In another preferred embodiment, the first strip-like separation media are arranged roughly parallel to each other on the first carrier and the second strip-like separation media are also arranged roughly parallel to each other on the second carrier.

The roughly parallel orientation of the separation media on their respective carriers allows a highly parallel processing of the separation media in one step because a high amount of these separation media can be easily processed even in a fully automated manner in one step. For example, it is possible to load the molecules to be separated in A) onto all the first separation media in an automated procedure using robots.

In B), the first and second separation media are preferably brought into direct contact. This allows an easy direct transfer of the molecules already pre-selected in A) from the first to the second separation media. If gel strips are used as first and second separation media this variant of the method enables an easy “gel to gel loading” of the molecules without any additional transfer steps like extraction of the molecules from the first separation media and subsequent transfer onto the second separation media.

Preferably in B), the second separation media are orientated diagonally to the first separation media, so that every second separation medium is in contact with more than one first separation medium.

This variant enables a fast and easy transfer of the molecules pre-separated in A) and located on different first separation media onto one single second separation medium.

In another preferred embodiment, in B) molecules of roughly the same isoelectric point range located on different first strip-like separation media are transferred to the same one second strip-like separation medium roughly having the same isoelectric point range as the molecules being transferred.

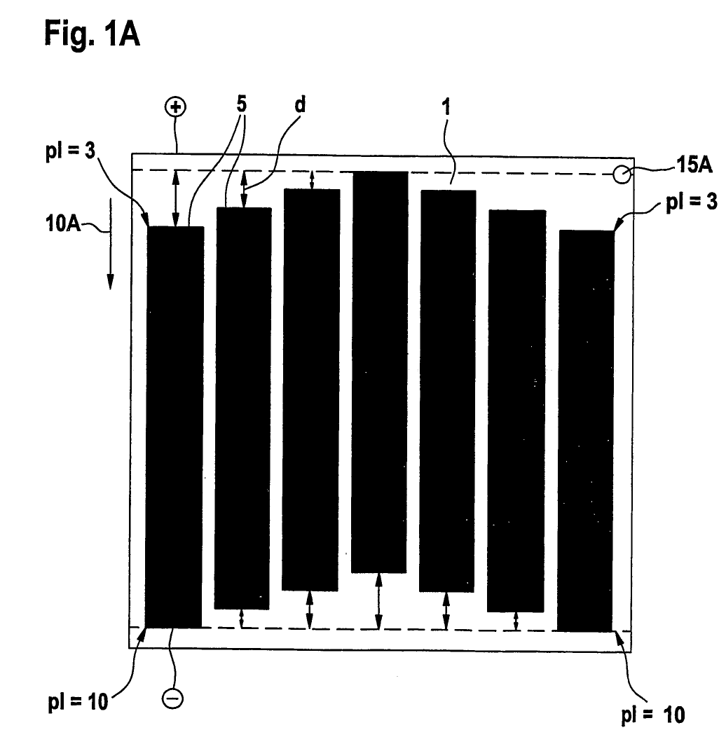

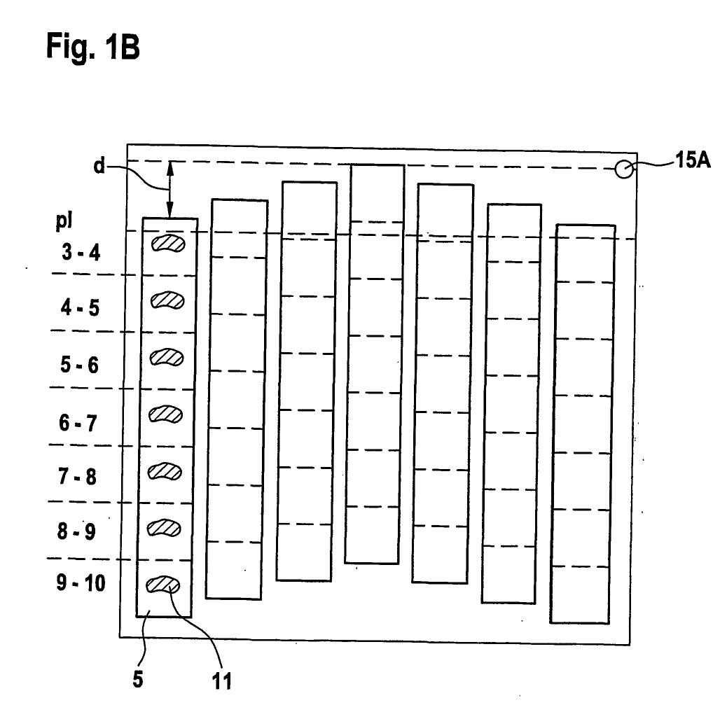

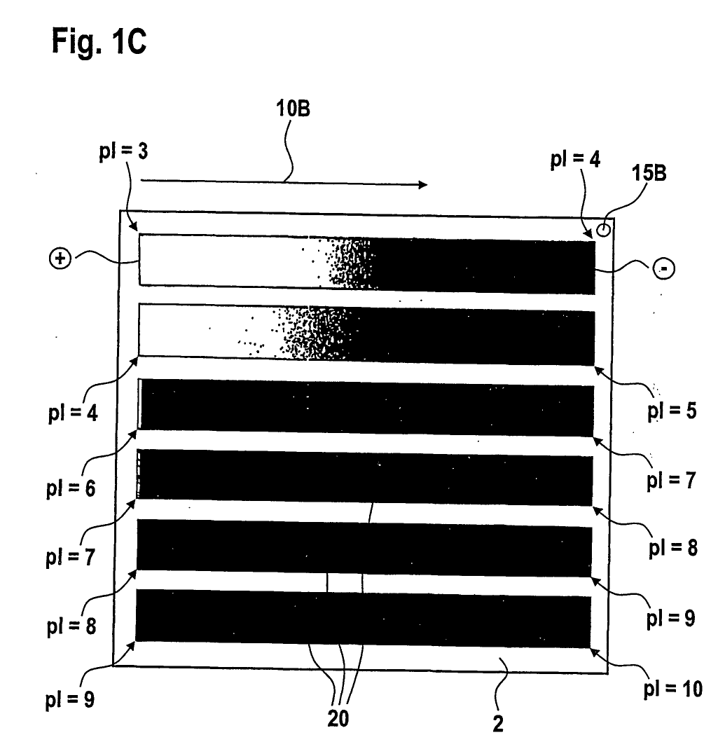

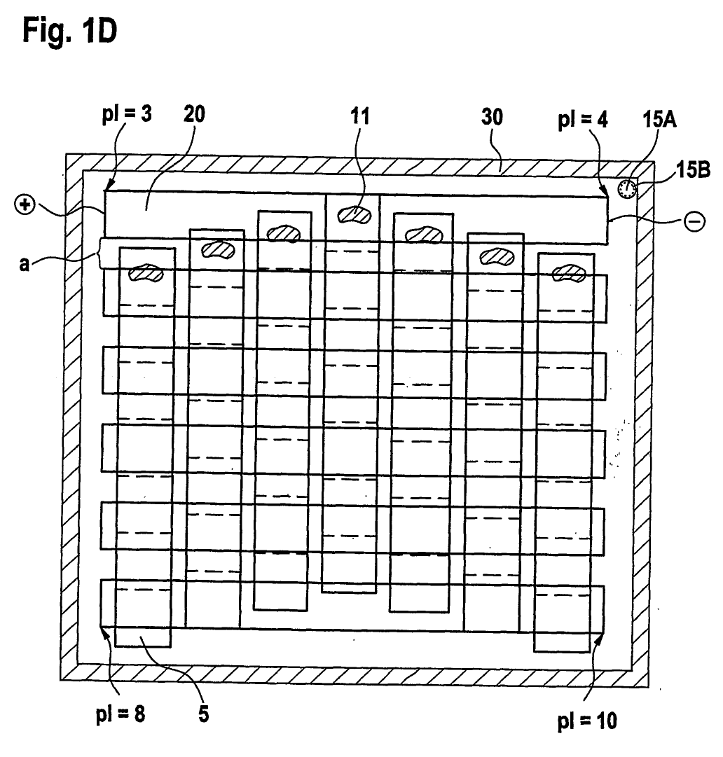

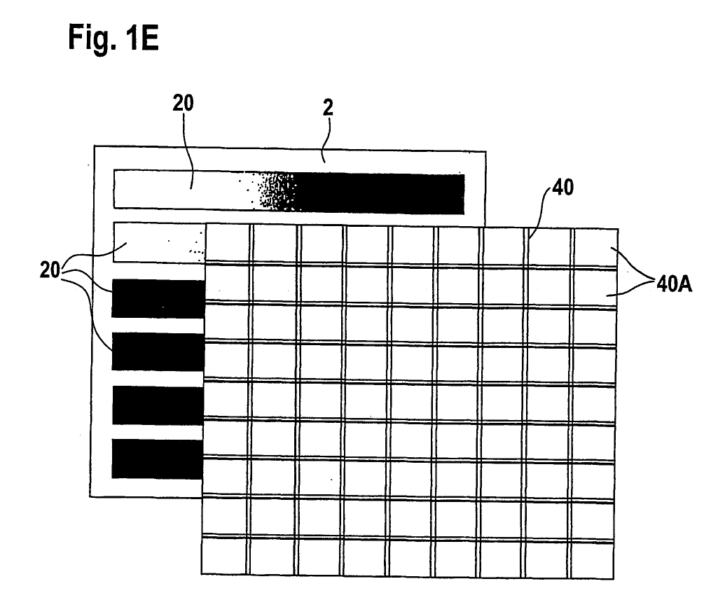

During the first wide range isoelectric focusing separation the molecules are normally pre-separated according to a relatively wide pl range e.g. from 3 to 10, resulting in a large pl gradient from the starting point of the first separation media to their end points. Therefore after the first IEF separation in A) different bands of molecules are present in the first separation media, each band containing different molecules with a. relatively brought distribution of isoelectric points, see for example FIG. 1b). In this case molecule bands which are for example located in pl areas within pl 3 and 4 might then preferably be transferred to a second separation medium which has a pl gradient from 3 to 4. The second separation media preferably might have the same length as the first separation media, so that the second separation media have a much lower gradient compared to the first separation media, allowing a separation with a higher resolution than in A). After this transfer B) the molecules with the pl ranges 3 to 4 will then be subjected to a further narrow range separation C) isolating molecule species with a difference in their pls of roughly 0.1 to 0.05 pl units.

Preferably, gel strips are used as first and second separation media. Gel strips are easy to handle and provide a good focusing ability especially for proteins.

Favorably in B) the first and second separation media are brought into direct contact and then a voltage is applied to the second separation media in order to transfer the molecules according to B) and carry out the separation according to C).

When the first and second separation media are in direct contact and a voltage is applied to the second separation media the molecules, especially in the case of proteins, easily diffuse from the first separation media into the second separation media due to electrokinetic and diffusion effects. This variant is an easy and fast approach because B) and C) are carried out in one step (see e.g. FIG. 1D).

After the second separation of the molecules in C) or alternatively after the first separation in A), a grid-like structure is preferably brought into contact with the second separation media in D2) or D1), thereby separating the first or second separation media into different compartments. Afterwards at least one solvent can be applied into the different compartments extracting the molecules.

After D1) or D2), the molecules separated by their isoelectric points are in liquid phase and can therefore easily be further processed and analyzed for example by using one-dimensional or multi-dimensional LC chromatography and subsequent analysis using mass-spectroscopy.

It is also possible that prior to application of the at least one solvent, the molecules in the first or second separation media are being fragmented/digested. Fragments of the molecules are normally easier to bring into a liquid phase than the complete molecules. This especially accounts for proteins, which can be digested with proteolytic enzymes like trypsin and can then be easier extracted from, e.g. gel strips as separation media.

It is also possible to carry out a gel electrophoresis after D1) or D2) for further separation of the molecules according to their mass. In this case the two-step isoelectric focusing procedure or alternatively the one-step isoelectric focusing procedure of embodiments provide a pre-selection for the subsequent gel electrophoresis, which is normally a sodium dodecyl sulphate polyacrylamide gel electrophoresis. Such a variant might provide a high pi resolution separation performance due to the different methods of separation, the isoelectric focusing and the subsequent gel electrophoresis and also might ensure a optimal loading of the narrow range fractions from C) to the sodium dodecyl sulphate polyacrylamide gel in order to allow the identification of low abundance proteins and PTMs (protein isoforms).

It is also possible to carry out a one-dimensional or multidimensional liquid chromatography (LC) for further separation of the molecules after D1) or D2). Multidimensional liquid chromatography normally uses at least two different chromatographic separation techniques, for example ion exchange chromatography, which separates the molecules according to their charges, reversed-phase chromatography, which provides a quasi-molecular size separation in which retention tends to increase with increasing molecular weight, size exclusion chromatography or affinity chromatography like the removal of immunoglobulins using protein A columns. Liquid chromatography has the great advantage that high sample loads can be applied onto the columns therefore allowing the separation and detection of low abundance proteins. Afterwards the molecules separated by the liquid chromatography might be analyzed by mass spectroscopy (MS).

In another embodiment, the molecules are analyzed by a chip array after D1) or D2). For example, protein bio-chips can be used, where e.g. antibodies specific for a marker protein are immobilized on the chip. These marker proteins can bind to the antibodies on the chip and can be further analyzed. These variants enable the detection of e.g. marker proteins for special diseases in cells or tissues of patients by e.g. high throughput analysis.

An apparatus for separation of amphoteric molecules according to their isoelectric points comprises:
a first carrier with a plurality of first strip-like separation media having a respective first pl range for a first separation step.

Preferably, the apparatus further comprises:
a second carrier with a plurality of second strip-like separation media having a respective second pi range different from the first pl range for a second separation step.

This apparatus is suited e.g. to carry out a variant of the separation method. Preferably, first and second separation media comprise gel strips, wherein the gel strips comprise immobilized ampholytes. Immobilized ampholytes can provide a highly stable pi gradient.

The first and second carrier preferably comprise a flat plate or sheet, which can provide a stable area for the first and second separation media. The flat plate can comprise any kind of material suitable for a carrier, for example plastic, polymer, ceramic and glass or any kind of combination thereof.

The first and second carrier advantageously also comprise bar code identifiers for sample tracking. The bar code identifiers ensure an easy identification of first and second carriers and are also suitable for highly automated procedures.

Preferably, the first and second strip-like separation media are arranged roughly parallel to each other on their respective carriers. Furthermore it is preferred, that the ends of the first separation media on the first carrier are offset relative to each other and that the first separation media have the same length. This means that the first separation media which are roughly arranged parallel to each other are displaced along their length, as for example shown in the FIGS. 1A and 1B. This positional offset increases the distribution of proteins of different pl ranges on the first separation media perpendicular to the length of the first separation media after the first separation in A). This increased distribution helps to avoid “pl gaps” during the transfer in B).

The apparatus preferably furthermore comprises a positioner for positioning the second carrier and the first carrier relative to each other on top of each other, thereby positioning the first strip-like separation media diagonally to the second strip-like separation media. This enables an easy diagonal transfer of most of the molecules separated in A) to the second separation media in B).

The positioner can preferably comprise a frame, which keeps the two carriers together and prevents slipping of the carriers during the transfer procedure B).

The positioner advantageously comprises a first positioning element on one carrier and a second positioning element mating to the first element on the other carrier, fitting into each other when the first separation media are orientated diagonally relative to the second separation media. The first positioning element and the second positioning element can for example be a protrusion on one carrier and an indentation with a complementary shape to the protrusion on the other carrier, as for example shown in the FIGS. 1B and 1C. These first and second positioning elements ensure, that the first separation media are orientated diagonally or even perpendicular to the second separation media during the transfer in B) (see for example FIG. 1D). These means of positioning provide an easy handling of the apparatus.

In the following, exemplary embodiments of the invention will be explained in more detail by the figures. All figures are just simplified schematic representations presented for illustration purposes only.

### BRIEF DESCRIPTION OF THE DRAWINGS

The FIGS. 1A to 1E depict top views of different separation media during various steps of embodiments of the invention.

### DETAILED DESCRIPTION OF EMBODIMENTS OF THE INVENTION

FIG. 1a shows a top view of a first carrier 1 with seven first strip-like separation media 5 formed of gel strips during A). The gel strips 5 are all positioned parallel to each other, being displaced along their length, thereby creating an offset d. The gel strips 5 all have the same pl range, for example between a pl of 3 and 10. The arrow 10A indicates the direction of the first isoelectric focusing procedure A) when a voltage is applied across the gel strips 5 as indicated with the plus and minus signs. Furthermore, a first positioning element 15A, for example in the form of a protrusion, is present on the first carrier. This element is useful for the right orientation of the first and second carrier relative to each other during the transfer in B) (see FIG. 1D).

FIG. 1B depicts the carrier of FIG. 1A after the first wide range separation was carried out in A). The protein bands 11 are separated along the length of the strips according to their pi ranges. Due to the large pl gradient of the first gel strips 5 a pre-separation with relatively low resolution was performed, so that each protein band 11 still might comprise different proteins with similar isoelectric points. When a cell lysate is applied on the first gel strips 5, one continuous band of proteins distributed over the entire length of the first gel strips might result after the first separation, due to the large amount of different proteins in the cell lysate. In order to simplify the presentation, distinctive consecutive protein bands 11 are shown in FIG. 1D for every pl range between 3 and 10 for just one first gel strip 5. This first wide range separation A) might be a pre-selection for a second isoelectric separation in C) or alternatively a pre-selection for further multidimensional liquid chromatography, gel electrophoresis or analysis by a chip array after the extraction in D2).

FIG. 1C shows a top view of a second carrier 2 with second gel strips 20 during the second separation in C). The arrow 10B denotes the direction of the second narrow range separation. A second positioning element 15B mating to the first positioning element 15A is present on the second carrier 2. The second positioning element 15B can, for example, be an indentation or even a hole fitting together with the protrusion 15A of the first carrier when the first 5 and second 20 gel strips are orientated perpendicular to each other and are in direct contact. For the second narrow range separation in C) the second gel strips 20 have different consecutive starting and end points for their pi ranges. Neighboring gel strips 20 have consecutive pi ranges, for example, one gel strip pl range 3 to 4, the next, neighboring gel strip 4 to 5 and so on as indicated in FIG. 1C. This special arrangement of second gel strips 20 enables an easy orthogonal transfer of the proteins separated in A) to the second gel strips.

FIG. 1D shows another embodiment carrying out B) and C) in one step. The first carrier 1 with the first gel strips 5 is located on top of the second carrier 2 with the second gel strips 20. A frame 30 keeps the two carriers together and prevents a displacement during the transfer of the molecules in B). Furthermore the two positioning elements 15A and 15B are fitting together, indicating that the second gel strips are orientated orthogonal to the first gel strips 5, so that every second gel strip 20 is in contact with all the first gel strips 5. In order to simplify the presentation in FIG. 1D, the protein bands 11 present in all first gel strips 5 are just shown for one pi range between 3 and 4.

Due to the positional offset d of the first gel strips 5 to each other, protein bands 11 located on slightly different areas of the first gel strips 5 are transferred to one second gel strip 20, thereby creating a “fuzziness” in the pl ranges in order to avoid “pl gaps” during the transfer in B) due to the distances a between adjacent second gel strips 20. As shown in FIG. 1 D protein bands 11 roughly located in the pl range 3 to 4 on the first gel strips 5 are mostly transferred to the second gel strip 20 having a similar pl gradient of 3 to 4. The transfer of the molecules from the first gel strips 5 to the second gel strips 20 and the second separation in C) can be carried out in one step when a voltage, indicated by the plus and minus signs, is applied to the second gel strips 20.

FIG. 1E depicts a top view of an arrangement of a second carrier 2 and a grid-like structure 40 during D2) of an embodiment. A bottomless microtiter plate 40 with different compartments 40A is shown. This microtiter plate 40 can be arranged over the second carrier 2 and pressed into the second gel strips 20, therefore separating the gel strips into different compartments, containing different proteins with different isoelectric points. Subsequently solvents can be applied to each compartment, extracting the proteins from the gel. The same extraction procedure can also be carried out after A) using the first separation media (D1).

The scope of the invention is not limited to the embodiments shown in the figures. Indeed, variations especially concerning the pi gradients for first and second separation media and the relative orientation of the separation media to each other are possible.

Embodiments of the invention can be embodied in each novel characteristic and each combination of characteristics, which includes every combination of any features, which are stated in the claims, even if this combination of features is not explicitly stated in the claims.

What is claimed is:

1. A method for electrophoretic separation of amphoteric molecules according to their isoelectric points, comprising:
A) carrying out a first separation according to the isoelectric points of the amphoteric molecules in one ore more first strip-like separation media located on a first carrier, wherein the first separation media have a respective first pl range,
B) transferring the amphoteric molecules separated A) to a plurality of second strip-like separation media located on a second carrier, wherein the second separation media have a respective second pl range different from the first pl range,
C) carrying out a second separation of the molecules in the plurality of second separation media according to isoelectric points, and
D2) isolating the molecules from the second strip-like separation media.

2. Method according to claim 1, further comprising:
D1) isolating the molecules separated A) from the first strip-like separation media.

3. (canceled)

4. Method according to claim 1,
wherein first separation media each having the same pl range are used,
wherein second separation media each having different starting- and end-points for its pl range are used.

5. Method according to claim 1,
wherein the first and second separation media are selected in such a way, that the pl range of the first separation media is larger than the pl range of the second separation media.

6. Method according to claim 1,
wherein in A) first separation media with a maximum pl range from 1 to 12 are used, and
wherein in C) second separation media with a maximum pl range <1 are used.

7. Method according to claim 1,
wherein the first strip-like separation media are arranged roughly parallel to each other on the first carrier, and
wherein the second strip-like separation media are arranged roughly parallel to each other on the second carrier.

8. Method according to claim 1,
wherein in B) the first and second separation media are brought into direct contact.

9. Method according to claim 8,
wherein in B) the second separation media are orientated diagonally to the first separation media, so that every second separation medium is in contact with more than one of the first separation media.

10. Method according to claim 1,
wherein in B) molecules of roughly the same isoelectric point range, located on different first strip-like separation media are transferred to a same one of the second strip-like separation media roughly having the same isoelectric point range as the molecules being transferred.

11. Method according to claim 1,
wherein gel-strips are used as first and second separation media.

12. Method according to claim 1,
wherein in B) the first and second separation media are brought into direct contact,
wherein a voltage is applied to the second separation media in order to transfer the molecules according to B) and carry out the separation according to C).

13. Method according to claim 1,
wherein in D2) a grid-like structure is brought into contact with the second separation media, thereby separating the second separation media into different compartments,
wherein at least one solvent is applied into the different compartments, extracting the molecules.

14. Method according to claim 1,
wherein prior to application of the at east one solvent, the molecules in the second separation media are being fragmented.

15. Method according to claim 2,
wherein after D1) or D2) a gel electrophoresis is carried out further separating the molecules according to their mass.

16. Method according to claim 2,
wherein after D1) or after D2) a one-dimensional or multidimensional liquid chromatography (LC) is conducted for further separation of the molecules,
wherein after the LC the molecules are analyzed by mass spectroscopy (MS).

17. Method according to claim 16,
wherein the one-dimensional or multidimensional liquid chromatography comprises at least one of the following separation techniques:
ion exchange chromatography, reversed phase chromatography, size exclusion chromatography, affinity chromatography.

18. Method according to claims 2,
wherein after D1) or after D2) the molecules are analyzed by a chip array.

19. Method according to claim 2,
wherein in D1) a grid-like structure is brought into contact with the first separation media, thereby separating the first separation media into different compartments,
wherein at least one solvent is applied into the different compartments, extracting the molecules.

20. An apparatus for a two step separation of amphoteric molecules according to their isoelectric points comprising:
a first carrier with one ore more first strip-like separation media having a respective first pi range for a first separation step; and
a second carrier with a plurality of second strip-like separation media having a respective second pl range different from the first pl range for a second separation step.

21. (canceled)

22. Apparatus according to claim 20,
wherein the first and second separation media comprise gel-strips.

23. Apparatus according to claim 22,
wherein the gel-strips comprise immobilized ampholytes.

24. Apparatus according to claim 20,
wherein the first separation media have the same pl range, and
wherein the second separation media have different consecutive starting- and end-points for their pl ranges.

25. Apparatus according to claim 20,
wherein the first strip-like separation media are arranged roughly parallel to each other on the first carrier, and
wherein the second strip-like separation media are arranged roughly parallel to each other on the second carrier.

26. Apparatus according to claim 25,
wherein the first separation media have the same length, and
wherein the ends of the first separation media are offset relative to each other.

27. Apparatus according to claim 20 further comprising
a positioner adapted for positioning the second carrier and the first carrier relative to each other on top of each other, thereby positioning the strip like first separation media diagonally to the strip like second separation media.

28. Apparatus according to claim 27,
wherein the positioner comprises a frame.

29. Apparatus according to claim 27,
wherein the positioner comprises a first positioning element on one carrier and a second positioning element mating to the first element on the other carrier, fitting into each other when the first separation media are orientated diagonally relative to the second separation media.

30. Apparatus according to claim 20 further comprising
a grid-like structure with different compartments for extraction of the molecules from the first or second separation media.

31. Apparatus according to claim 30,
wherein the grid-like structure comprises a bottomless frame with separate compartments.

32. Apparatus according to claim 20,
wherein the first and second carrier comprise a flat plate or sheet.

33. Apparatus according to claim 20,
wherein the first and second carrier comprise bar code identifiers for sample tracking.

34. An apparatus, for separation of amphoteric molecules according to their isoelectric points, adapted for
A) carrying out a first separation according to the isoelectric points of the amphoteric molecules in one ore more first strip-like separation media located on a first carrier, wherein the first separation media have a respective first pl range,
B) transferring the amphoteric molecules separated A) to a plurality of second strip-like separation media located on a second carrier, wherein the second separation media have a respective second pl range different from the first pl range,
C) carrying out a second separation of the molecules in the plurality of second separation media according to isoelectric points, and
D) isolating the molecules from the second strip-like separation media.
